(19) United States
(12) Patent Application Publication
(10) Pub. No.: US 2010/0249818 A1
(43) Pub. Date: Sep. 30, 2010

(54) METHOD OF MANUFACTURING MEDICAL INSTRUMENT AND MEDICAL INSTRUMENT

(75) Inventors: Makoto Jinno, Ashigarakami-gun (JP); Yoshiaki Yuno, Fujinomiya-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: TERUMO KABUSHIKI KAISHA, Shibuya-ku (JP)

(21) Appl. No.: 12/722,199

(22) Filed: Mar. 11, 2010

(30) Foreign Application Priority Data
Mar. 27, 2009 (JP) 2009-080479

(51) Int. Cl. A61B 17/32; B23P 11/00

(52) U.S. Cl. 606/174; 29/428

(57) ABSTRACT

Scissors on the distal end of a medical instrument are assembled highly accurately for cutting an object with ease. A scissors mechanism is assembled as a unit. The scissors mechanism has a pair of end effector members openably and closably fastened at proximal ends thereof by a bolt and nuts, while being held in a predetermined sliding state. The bolt has a central hole defined axially therethrough. The scissors mechanism is inserted into a tubular structure of a cover, which is coupled to the distal end of a joint shaft, and the end effector members are connected to a driven plate by links. A spacer is placed in a gap between the scissors mechanism and an inner surface of the cover. A pin is inserted, from an outer surface of the cover, into a hole of the cover and the central hole of the bolt.

### CROSS-REFERENCE TO RELATED APPLICATION

This application is based upon and claims the benefit of priority from Patent Application No. 2009-080479 filed on Mar. 27, 2009, in the Japan Patent Office, of which the contents are incorporated herein by reference.

### BACKGROUND OF THE INVENTION

1. Field of the Invention

The present invention relates to a method of manufacturing a medical instrument having a pair of scissors on the distal end of a shaft for cutting off a portion of a living body, a thread, etc. The invention also relates to such a medical instrument itself.

2. Description of the Related Art

According to a laparoscopic surgical operation process, a certain number of small holes are opened in the abdominal region, for example, of a patient, and an endoscope and manipulators or forceps are inserted into the holes. The surgeon performs a surgical operation on the patient with the manipulators or forceps while watching an image captured by the endoscope and displayed on a display monitor. Since the laparoscopic surgical operation process does not require a laparotomy, the surgical operation is less burdensome on the patient and greatly reduces the number of days required for the patient to spend before recovering from the operation and being released from the hospital. Therefore, the laparoscopic surgical operation process is expected to find an increased range of surgical operations to which it is applicable.

Manipulators for laparoscopic surgical operations are required to allow the operator, i.e., the surgeon, to perform various appropriate techniques quickly depending on the position and size of the affected part, for removing, suturing, and ligating the affected part. The present applicants have proposed manipulators, which can be manipulated easily with a high degree of freedom (see, for example, Japanese Laid-Open Patent Publication No. 2002-102248, Japanese Laid-Open Patent Publication No. 2008-104854, and Japanese Laid-Open Patent Publication No. 2008-253463).

In laparoscopic surgical operations, cutting processes for removing an affected part of the patient and cutting off suture threads are performed. A manipulator with scissors on a distal end thereof has been developed for carrying out such cutting processes (see, for example, Japanese Laid-Open Patent Publication No. 10-314178).

As known in the art, a pair of scissors cuts an object by applying a shearing force to the object from a pair of cutting blades. For applying an effective shearing force to the object, it is desirable that the cutting blades be sufficiently adjusted to hold their cutting edges slidably against each other with no gap therebetween.

Medical manipulators have a distal-end working unit which is extremely small. If a scissors mechanism is to be installed on the tip end of such a distal-end working unit, then the scissors mechanism is difficult to assemble, and the worker needs to be highly skilled in order to assemble the cutting edges of the scissors mechanism accurately on the distal-end working unit with no gap between the cutting edges.

### SUMMARY OF THE INVENTION

It is an object of the present invention to provide a method of manufacturing a medical instrument having a scissors mechanism and which is assembled highly accurately for easily cutting off an object. A further object of the present invention is to provide such a medical instrument itself.

According to the present invention, a method of manufacturing a medical instrument comprises a first step of superposing a pair of openable and closable members one on each other, and inserting a shank of a bolt through holes defined in proximal ends of the openable and closable members, the bolt having a shaft hole defined axially through the bolt, a second step of threading a nut on the shank of the bolt, a third step of sandwiching the superposed openable and closable members between a head of the bolt and the nut, and while performing a predetermined sliding adjustment process on the superposed openable and closable members, securing the bolt and the nut to each other to produce a structural body in which the openable and closable members are angularly movably supported in a predetermined sliding state, a fourth step of inserting the structural body into a connecting tube coupled to a distal end of a shaft extending from an operating unit, and connecting the openable and closable members to a transmitting member for transmitting an input action from the operating unit to the openable and closable members, and a fifth step of fitting and securing a pin, from an outer surface of the connecting tube, in a hole defined diametrically through the connecting tube and the shaft hole of the bolt, whereby the openable and closable members are angularly movably supported on the shank of the bolt for opening and closing movement about the shank.

The first, second, and third steps do not need to be carried out in a small space, and there are no interlinking members involved in the first, second, and third steps. Therefore, the structural body can be assembled highly accurately. In the fourth and fifth steps, the pin is fitted into the shaft hole of the bolt in order to assemble the structural body into the connecting tube. The structural body and the connecting tube can thus be assembled together easily, while the openable and closable members of the structural body are kept in a sliding state highly accurately.

The method may include, after the fourth step and before the fifth step, a spacer insertion step of placing a spacer in a gap between the structural body and an inner surface of the connecting tube. The spacer, which is placed in the gap, makes it possible for the structural body having the fixed openable and closable members to be adjusted in position and secured in the connecting tube.

A medical instrument according to the present invention comprises a structural body including a pair of superposed openable and closable members fastened to each other in a predetermined sliding state by a bolt and a nut, the superposed openable and closable members being openable and closable about a shank of the bolt, a connecting tube housing the structural body therein and coupled to a distal end of a shaft extending from an operating unit, a pin fitted into a shaft hole defined axially through the bolt in the structural body which is housed in the connecting tube, the openable and closable members being angularly movably supported by the pin, and a transmitting member for transmitting an input action from the operating unit to the openable and closable members.

The openable and closable members are angularly movably supported by the pin while being kept in a predetermined sliding state. The structural body can easily be assembled using the shaft hole, which is defined axially through the bolt.

The medical instrument may further comprise a spacer disposed in the connecting tube and placed in a gap between the structural body and an inner surface of the connecting tube, the pin being inserted in the spacer. The spacer, which is placed in the gap, makes it possible to prevent the structural body having the fixed openable and closable members from being displaced within the connecting tube.

The openable and closable members may comprise a pair of scissors held in sliding contact with each other for cutting an object.

With the method of manufacturing a medical instrument and the medical instrument according to the present invention, since the first, second, and third steps do not need to be carried out in a small space, and since there are no interlinking members involved in the first, second, and third steps, the structural body can be assembled highly accurately. In the fourth and fifth steps, the pin is fitted into the shaft hole of the bolt in order to assemble the structural body into the connecting tube. The structural body and the connecting tube can thus be assembled together easily, while the openable and closable members of the structural body are kept in a sliding state highly accurately.

The above and other objects, features, and advantages of the present invention will become more apparent from the following description when taken in conjunction with the accompanying drawings in which preferred embodiments of the present invention are shown by way of illustrative example.

### BRIEF DESCRIPTION OF THE DRAWINGS

FIG. 1 is a side elevational view of a manipulator according to an embodiment of the present invention;

FIG. 2 is a plan view of the manipulator shown in FIG. 1;

FIG. 3 is a schematic side elevational view of a distal-end working unit of the manipulator with a trigger lever being fully pulled;

FIG. 4 is a schematic side elevational view of the distal-end working unit of the manipulator with the trigger lever being pushed out;

FIG. 5 is a schematic perspective view showing structural details of the distal-end working unit;

FIG. 6 is a sectional side-elevational view of the distal-end working unit;

FIG. 7 is a sectional plan view of the distal-end working unit;

FIG. 8 is a sectional side-elevational view of the distal-end working unit with a gripper being closed;

FIG. 9 is an exploded perspective view of the distal-end working unit;

FIG. 10 is a schematic perspective view showing structural details of an end effector drive mechanism;

FIG. 11 is a schematic side elevational view of the end effector drive mechanism at a time when the trigger lever is not operated;

FIG. 12 is a sectional plan view of a portion of a second end effector drive mechanism at a time when the trigger lever is pushed out;

FIG. 13 is a sectional plan view of a portion of the second end effector drive mechanism at a time when the trigger lever is fully pulled;

FIG. 14 is a sectional side elevational view of a portion of the second end effector drive mechanism at a time when the trigger lever is pushed out;

FIG. 15 is a perspective view of a scissors mechanism;

FIG. 16 is an exploded perspective view of the scissors mechanism;

FIG. 17 is a fragmentary cross-sectional view showing a first stage for assembling the distal-end working unit;

FIG. 18 is a fragmentary cross-sectional view showing a second stage for assembling the distal-end working unit;

FIG. 19 is a fragmentary cross-sectional view showing a third stage for assembling the distal-end working unit;

FIG. 20 is a fragmentary cross-sectional view showing a fourth stage for assembling the distal-end working unit;

FIG. 21 is a fragmentary cross-sectional view showing a fifth stage for assembling the distal-end working unit;

FIG. 22 is a fragmentary cross-sectional view showing a sixth stage for assembling the distal-end working unit;

FIG. 23 is a fragmentary cross-sectional view showing a first stage for assembling a distal-end working unit according to a first modification of the present invention;

FIG. 24 is a fragmentary cross-sectional view showing a second stage for assembling the distal-end working unit according to the first modification of the present invention;

FIG. 25 is a fragmentary cross-sectional view showing a third stage for assembling the distal-end working unit according to the first modification of the present invention;

FIG. 26 is a schematic side elevational view showing structural details of a distal-end working unit according to a second modification of the present invention;

FIG. 27 is a schematic perspective view of a surgical robot system with a working unit connected to the distal end of a robot arm; and

FIG. 28 is a side elevational view of a pair of forceps.

### DESCRIPTION OF THE PREFERRED EMBODIMENTS

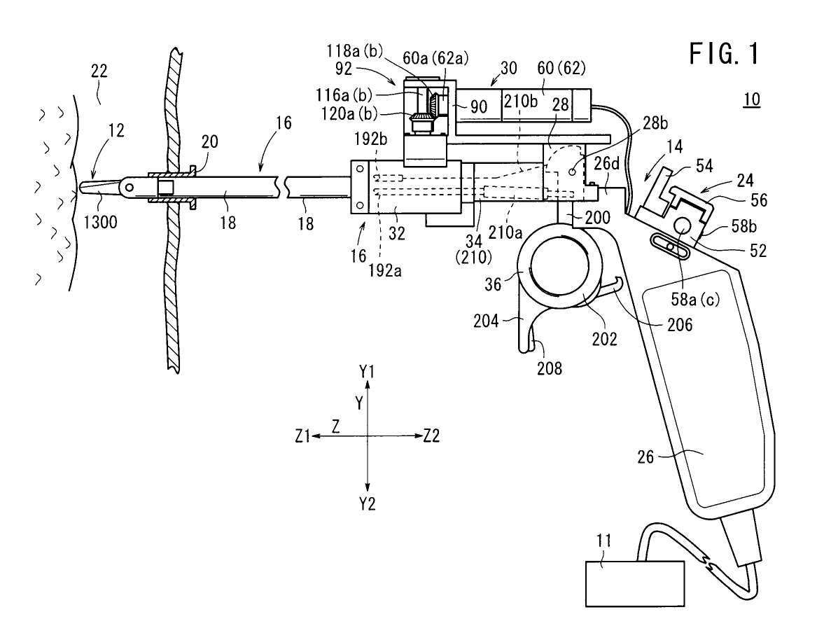

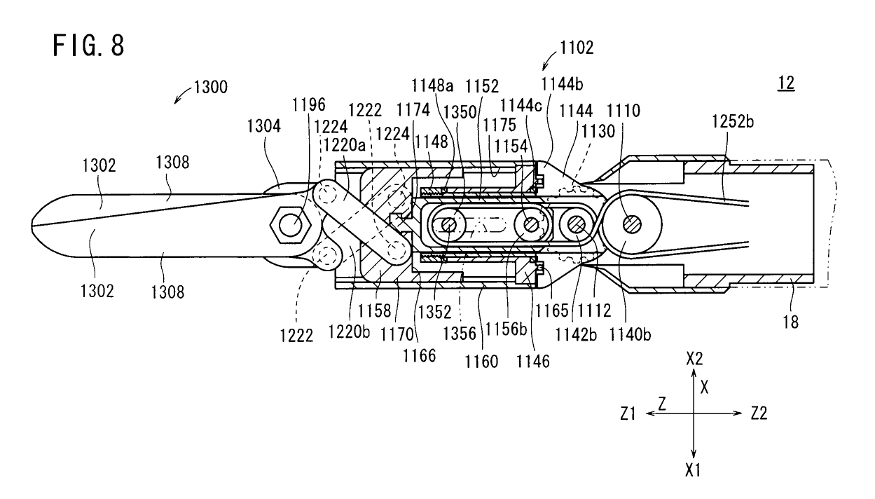

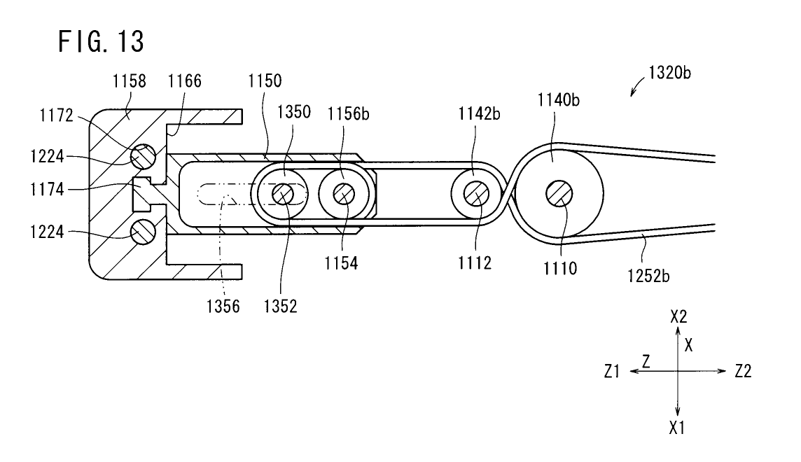

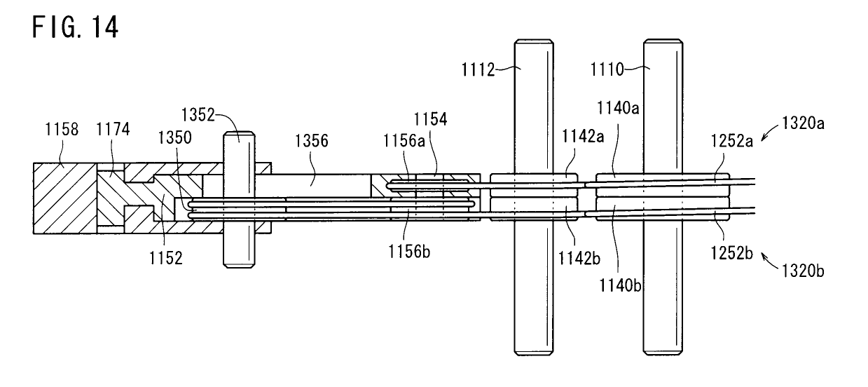

Manipulators according to embodiments of the present invention will be described below with reference to FIGS. 1 through 28.

As shown in FIG. 1, a manipulator 10 according to an embodiment of the present invention is electrically connected to a controller 11. The manipulator 10 is basically a medical manipulator for use in surgical operations.

The controller 11, which electrically controls the manipulator 10, is connected by a connector to a cable, which extends from the lower end of a grip handle 26 of the manipulator 10. The controller 11 is capable of independently controlling a plurality of manipulators 10 at the same time, although the controller 11 also can control a single manipulator 10, as shown in FIG. 1.

The manipulator 10 includes a distal-end working unit 12 having on its tip end a scissors mechanism (structural body) 1300 for cutting off a portion of a living body or a suture.

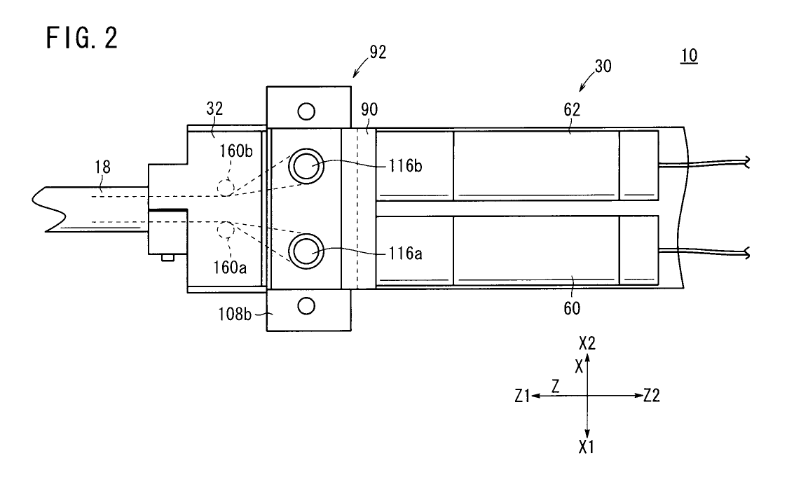

As shown in FIGS. 1 and 2, the manipulator 10 includes an operating unit 14 which is gripped and operated by a user's hand, and a working unit 16 fixed to the operating unit 14. The working unit 16 has a distal-end working unit 12 for performing a working process on a patient, and an elongate hollow joint shaft 18 that connects the distal-end working unit 12 and the operating unit 14 to each other. The distal-end working unit 12 and the joint shaft 18 have small diameters and can be inserted into a body cavity 22 through a trocar 20 in the form of a hollow cylinder mounted in an abdominal region or the like of the patient. The distal-end working unit 12 is actuated by a composite input unit 24 of the operating unit 14 in order to perform various techniques, such as removal of an affected part from the body cavity 22, or cutting off a thread or suture, etc. The operating unit 14 and the working unit 16 are integrally connected to each other. However, the operating unit 14 and the working unit 16 may also be connected to each other in a detachable manner.

In the description that follows, it is assumed that the transverse directions in FIGS. 1 and 2 are referred to as X directions, vertical directions as Y directions, and longitudinal directions of the joint shaft 18 as Z directions. Among the X directions, the rightward direction as viewed from the distal end is referred to as an X1 direction, and the leftward direction as an X2 direction. Among the Y directions, the upward direction is referred to as a Y1 direction, and the downward direction as a Y2 direction. Among the Z directions, the forward direction is referred to as a Z1 direction, and the rearward direction as a Z2 direction. Unless otherwise noted, these directions represent directions of the manipulator 10 when the manipulator 10 is in a neutral attitude. The definitions of the above directions are for illustrative purposes only. The manipulator 10 can be used in any of various orientations, e.g., it may be used upside down.

The operating unit 14 includes a grip handle 26 which is gripped by a hand, a bridge 28 that extends from an upper portion of the grip handle 26, and an actuator block 30 connected to a distal end of the bridge 28. The grip handle 26 has a length suitable for being gripped by the hand. The grip handle 26 includes the composite input unit 24, which is disposed on an upper slanted surface thereof. The grip handle 26 extends substantially in the Y2 direction from the end of the bridge 28. The grip handle 26, which extends in this manner, allows the user to handle the manipulator 10 easily when the manipulator 10 is moved as a whole, and permits the composite input unit 24 mounted on the upper slanted surface of the grip handle 26 to be operated easily by the user.

The working unit 16 comprises a pulley box 32 connected to the actuator block 30, the joint shaft 18 extending in the Z1 direction from the pulley box 32, the distal-end working unit 12 mounted on the distal end of the joint shaft 18, a support box 34 extending in the Z2 direction from the pulley box 32 to the bridge 28, and a trigger lever 36 pivotally supported on the proximal end of the support box 34 and which is joined to the bridge 28.

The distal-end working unit 12 is capable of moving about three axes based on actions made by the user using the composite input unit 24 and the trigger lever 36. More specifically, the distal-end working unit 12 is tiltable about a yaw-axis, which extends along the Y directions, is rotatable about a roll-axis, which extends toward the distal end of the working unit 16 (along the Z directions when the manipulator 10 is in a neutral attitude), and is openable and closable about a scissors-axis. The distal-end working unit 12 is tilted about the yaw-axis and is rotatable about the roll-axis by motors 60, 62 when electric switches, not shown, associated respectively with a yaw-axis input device 56 and a roll-axis input device 54 are turned on, and when the user moves the yaw-axis input device 56 and the roll-axis input device 54 laterally to the left or right. At this time, the motors 60, 62 operate as a roll-axis actuator and/or a yaw-axis actuator. The distal-end working unit 12 comprises a scissors mechanism 1300, which is mechanically driven about the scissors-axis when the trigger lever 36 is operated by the user.

The composite input unit 24 comprises a base block (not shown), a housing 52 mounted on the base block, the roll-axis input device 54, the yaw-axis input device 56, and three switch operators 58a, 58b, 58c. When the trigger lever 36 is pulled, a rod 192a coupled thereto also is pulled in unison therewith. When the trigger lever 36 is pushed and pulled, the rod 192a and a rod 192b, which also is coupled to the trigger lever 36, are operated. Although no initial position setting is established for the trigger lever 36, the trigger lever 36 may be set in a non-operative initial attitude by a resilient member, not shown, and may be closed when pulled toward the grip handle 26.

As shown in FIGS. 1 and 2, the actuator block 30 includes the two motors 60, 62, an actuator bracket 90 on which the motors 60, 62 are supported, and a gear mechanism 92 for transmitting rotational forces of the motors 60, 62 to the working unit 16, while changing directions of rotation of the motors 60, 62. The actuator bracket 90 is connected to the distal end of the bridge 28.

The motors 60, 62 each have a cylindrical shape and are supported on the actuator bracket 90, such that the motors 60, 62 extend in the Z directions and are juxtaposed in the X directions. The motors 60, 62 have respective output shafts 60a, 62a projecting from one end thereof in the Z1 direction.

The gear mechanism 92 is disposed in a space surrounded by three plates of the actuator bracket 90, which extend in the Z1 direction. The gear mechanism 92 is symmetrical in structure in the X directions.

The gear mechanism 92 comprises two drive shafts 116a, 116b, two drive bevel gears 118a, 118b, and two driven bevel gears 120a, 120b.

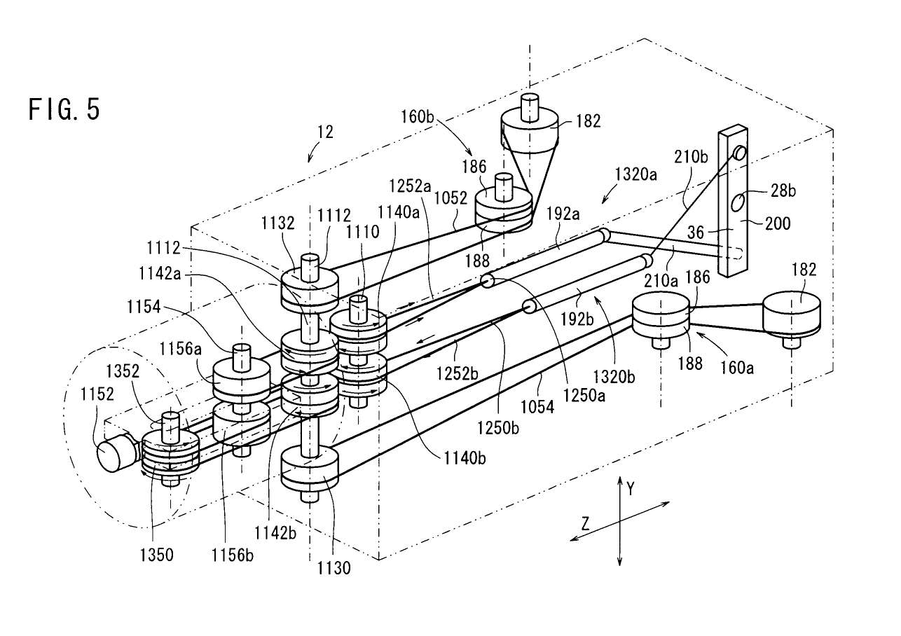

The drive shafts 116a, 116b have upper ends and central portions rotatably supported by bearings, and lower ends that project through shaft holes in the Y2 direction and extend into the pulley box 32. Wires 1052, 1054 (see FIG. 5) are trained around respective pulleys 182 mounted on the drive shafts 116a, 116b and extend around respective wire guides 160a, 160b, to be described later, through a space in the joint shaft 18 and into the distal-end working unit 12. The wires 1052, 1054 may be of the same type and diameter.

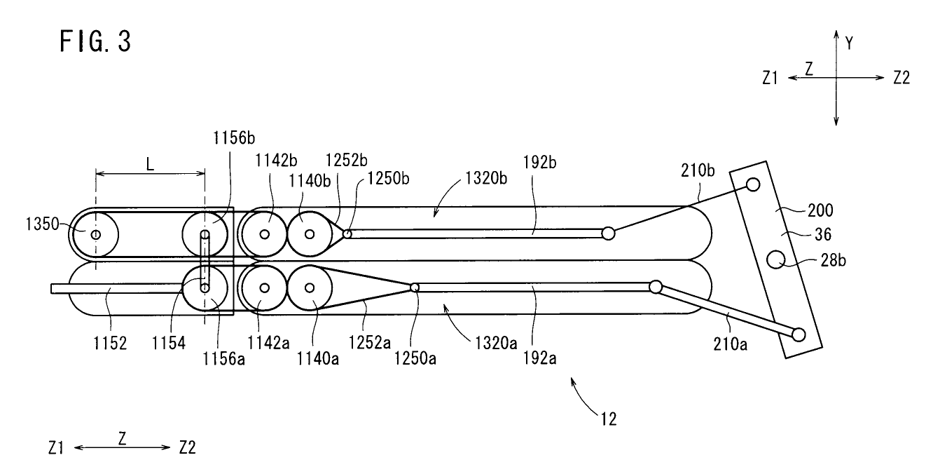

When the trigger lever 36 is manually pulled by the user, movement of the trigger lever 36, caused by a manual action from the user, is mechanically transmitted through the joint shaft 18 to the scissors mechanism 1300 in order to open or close the scissors mechanism 1300. Between the trigger lever 36 and the scissors mechanism 1300, there is disposed an action transmitting mechanism, which includes a load limiter 210a, a trigger wire 210b, a rod 192a, and first and second end effector drive mechanisms 1320a, 1320b (see FIGS. 3 through 5), which jointly serve as a means for mechanically transmitting manual actions from the user.

The drive bevel gear 118a and the driven bevel gear 120a are held in mesh with each other, and transmit rotation of the output shaft 60a to the drive shaft 116a while converting the direction of rotation through 90°. Similarly, the drive bevel gear 118b and the driven bevel gear 120b are held in mesh with each other, and transmit rotation of the output shaft 62a to the drive shaft 116b while converting the direction of rotation through 90°.

The pulley box 32, which is connected to the gear mechanism 92 and the support box 34, has a first function to relay rotation of the drive shafts 116a, 116b to the joint shaft 18, a second function to relay movement of the trigger lever 36 to the joint shaft 18, and a third function to keep the space in the joint shaft 18 hermetically sealed.

The pulley box 32 houses the wire guides 160a, 160b therein. The wire guides 160a, 160b have cylindrical idlers 186, 188 (see FIG. 5) around which the wires 1052, 1054 are trained and extend into the joint shaft 18.

Structural details of the support box 34 and the trigger lever 36 will be described below.

As shown in FIG. 1, the trigger lever 36 is pivotally supported on the bridge 28 by a trigger shaft 28b. The trigger lever 36 includes an arm 200 pivotally mounted on the trigger shaft 28b, a finger ring 202 joined to the lower end of the arm 200 in the Y2 direction, a finger keeper 204 joined to the lower end of the finger ring 202 in the Y2 direction, and a ratchet 206 that projects from the finger ring 202 in the Z2 direction. The index finger of a hand that grips the grip handle 26 is inserted into the finger ring 202, whereas the middle and ring fingers of the hand are placed on the finger keeper 204.

The support box 34 has a support casing 210 disposed between the pulley box 32 and the trigger lever 36.

The support casing 210 houses therein the load limiter 210a and the trigger wire 210b, which connect the respective rods 192a, 192b to the arm 200. More specifically, the load limiter 210a connects the rod 192a to a portion of the arm 200 below the trigger shaft 28b, and the trigger wire 21b connects the rod 192b to a portion of the arm 200 above the trigger shaft 28b.

Structural details of the distal-end working unit 12 will be described below.

As shown in FIG. 3, the distal-end working unit 12 includes the first and second end effector drive mechanisms 1320a, 1320b. The first end effector drive mechanism 1320a includes the rod 192a, a driven wire 1252a, an idle pulley 1140a, a guide pulley 1142a, and a driven pulley 1156a. The second end effector drive mechanism 1320b includes the rod 192b, a driven wire 1252b, an idle pulley 1140b, a guide pulley 1142b, and a driven pulley 1156b. The first end effector drive mechanism 1320a and the second end effector drive mechanism 1320b make up basic mechanisms for opening and closing the scissors mechanism 1300.

Components of the first end effector drive mechanism 1320a are denoted by reference numerals with a suffix “a”, whereas components of the second end effector drive mechanism 1320b are denoted by reference numerals with a suffix “b”. Since certain components of the first end effector drive mechanism 1320a and the second end effector drive mechanism 1320b operate identically, only the identically operating components of the first end effector drive mechanism 1320a will be described below.

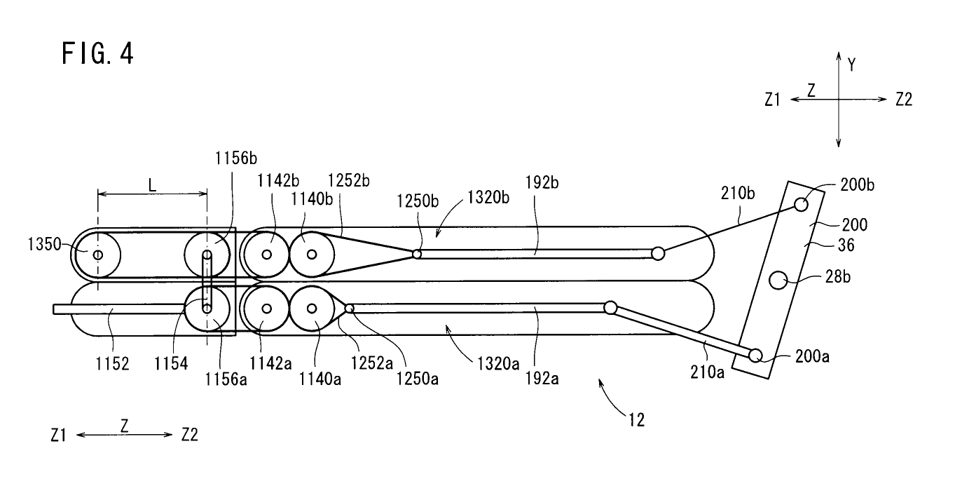

In FIGS. 3 and 4, the first end effector drive mechanism 1320a and the second end effector drive mechanism 1320b are shown as being juxtaposed in plan. In the actual manipulator 10, however, as shown in FIG. 5, the first end effector drive mechanism 1320a and the second end effector drive mechanism 1320b are juxtaposed in the axial directions of the pulleys (i.e., in the Y directions), with the idle pulleys (transmitting members) 1140a, 1140b being coaxial with each other, and the guide pulleys (transmitting members) 1142a, 1142b also being coaxial with each other. Therefore, the idle pulleys 1140a, 1140b are rotatably supported on a common shaft 1110 (see FIG. 5), and the guide pulleys 1142a, 1142b are rotatably supported on a common shaft 1112. Since the guide pulleys 1142a, 1142b are coaxial with each other, the manipulator 10 is tiltable about the yaw-axis by means of a simple mechanism.

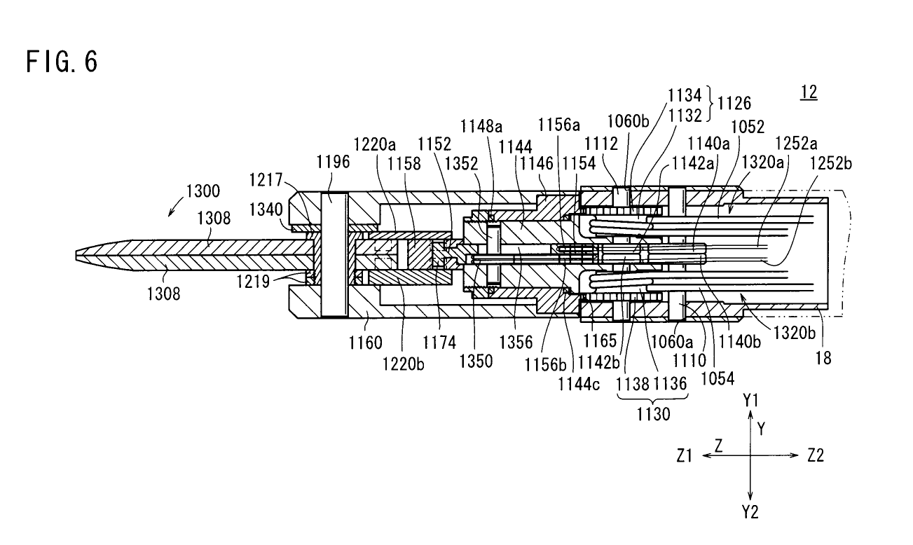

As shown in FIGS. 6 through 9, the distal-end working unit 12 comprises a wire-driven mechanism 1100, a composite mechanism 1102, and the scissors mechanism 1300, which collectively make up a unit. The distal-end working unit 12 incorporates therein mechanisms having three degrees of freedom. Such mechanisms include a mechanism having a first degree of freedom for angularly moving a portion of the distal-end working unit 12, which is positioned ahead of a first rotational axis Oy extending along the Y directions, in yawing directions about the first rotational axis Oy, a mechanism having a second degree of freedom for angularly moving the portion of the distal-end working unit 1012 in rolling directions about a second rotational axis Or, and a mechanism having a third degree of freedom for opening and closing the scissors mechanism 1300, which is disposed on the distal end of the distal-end working unit 12, about a third rotational axis Og.

The first rotational axis Oy of the mechanism with the first degree of freedom may be angularly moved out of parallel with an axis C, which extends from the proximal end toward the distal end of the joint shaft 18. The second rotational axis Or of the mechanism with the second degree of freedom may be angularly moved about an axis along the direction in which the distal end (scissors mechanism 1300) of the distal-end working unit 1012 extends, with the distal end portion thereof being rotatable in the rolling directions.

The mechanism with the first degree of freedom (i.e., movable in the yawing directions) comprises a tilting or bending mechanism having an operable range of ±90° or greater, for example. The mechanism with the second degree of freedom (i.e., movable in the rolling directions) comprises a rotating mechanism having an operable range of ±180° or greater, for example. The mechanism with the third degree of freedom (i.e., the scissors mechanism 1300) comprises an opening and closing mechanism openable through 40° or greater, for example.

The scissors mechanism 1300 constitutes a member for performing an actual cutting process during a surgical operation. The first rotational axis Oy and the second rotational axis Or make up attitude axes of an attitude changing mechanism, for changing the attitude of the scissors mechanism 1300 and facilitating the cutting process. Generally, the mechanism with the third degree of freedom for opening and closing the scissors mechanism 1300 is referred to as a gripper axis. The mechanism with the first degree of freedom for turning in the yawing directions is referred to as a yaw axis. The mechanism with the second degree of freedom for turning in the rolling directions is referred to as a roll axis.

The wire-driven mechanism 1100 is disposed between a pair of tongues 1058. The wire-driven mechanism 1100 serves to convert reciprocating movements of respective wires 1052, 1054 into rotational movements and to transmit the rotational movements to the composite mechanism 1102. The wire-driven mechanism 1100 includes one shaft 1110 inserted in shaft holes 1060a, 1060a, and another shaft 1112 inserted in shaft holes 1060b, 1060b. The shafts 1110, 1112 are press-fitted or welded securely in the shaft holes 1060a, 1060b. The shaft 1112 is axially aligned with the first rotational axis Oy.

Gear bodies 1126, 1130, which are symmetrically shaped in the Y directions, are mounted respectively on both ends of the shaft 1112 in the Y directions. The gear body 1126 comprises a tubular member 1132, and a gear 1134 disposed concentrically on an upper portion of the tubular member 1132. The gear body 1130 essentially is identical in shape to the gear body 1126, and is aligned with the gear body 1126 in the Y directions. The gear body 1130 comprises a tubular member 1136, and a gear 1138 disposed concentrically on a lower portion of the tubular member 1136. The gears 1134, 1138 are held in mesh with upper and lower ends of a face gear 1165 of a gear body 1146, which shall be described later.

The tubular member 1136 is substantially identical in diameter and shape to the tubular member 1132. The wires 1052, 1054 (see FIG. 5) are wound around the tubular members 1132, 1136, and have portions fastened to the tubular members 1132, 1136 by a given securing means. The wires 1052, 1054 are wound 1.5 turns (540°) around the tubular members 1132, 1136.

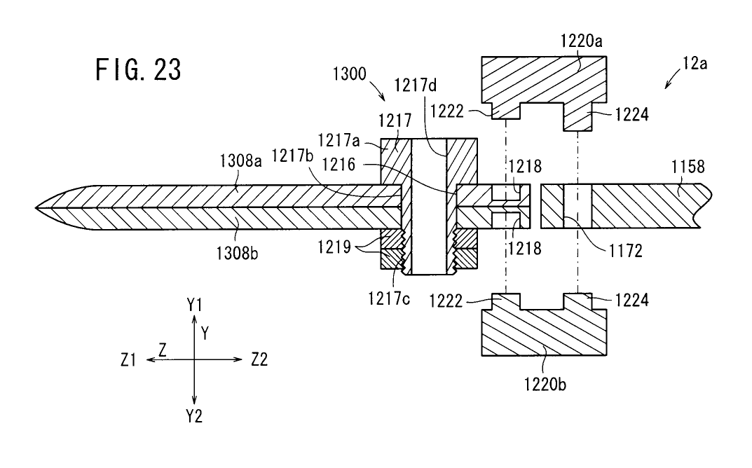

When the wires 1052, 1054 are rotated, the gear bodies 1126, 1130 are rotated about the shaft 1112. When the gear bodies 1126, 1130 are rotated at the same speed and in the same direction, the gear body 1146 swings with respect to the shaft 1112 and moves in the yawing directions. When the gear bodies 1126, 1130 are rotated at the same speed but in the opposite directions, the gear body 1146 is rotated about the second rotational axis Or and moves in the rolling directions. When the gear bodies 1126, 1130 are rotated at different speeds, the gear body 1146 makes a composite motion in both yawing and rolling directions. The gear body 1126, the gear body 1130, and the gear body 1146 make up a differential mechanism (corresponding to the structure shown in FIG. 23 of Japanese Laid-Open Patent Publication No. 2008-253463, for example).

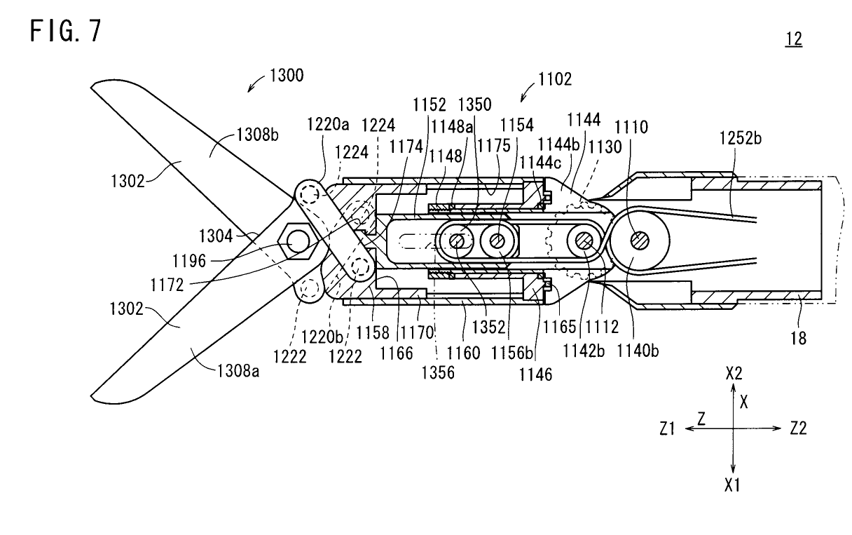

The mechanism of the distal-end working unit 12 is not limited to a differential mechanism, but may be a mechanism in which the wire 1052 causes the gear 1134 to actuate the face gear 1165, and the wire 1054 directly rotates a main shaft 1144 (corresponding to the structure shown in FIG. 7 of Japanese Laid-Open Patent Publication No. 2008-253463, for example).

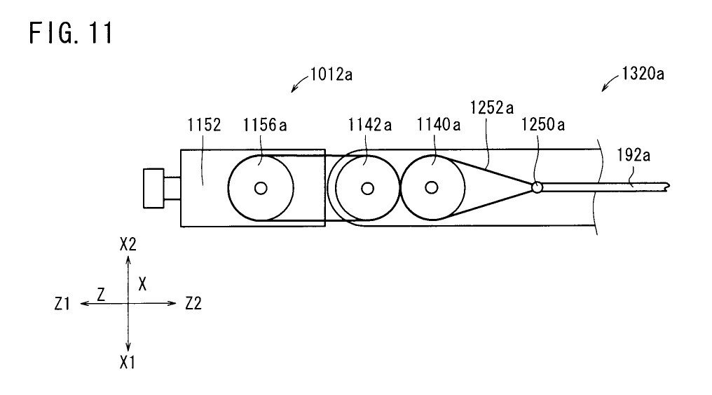

An idle pulley 1140a is rotatably supported substantially centrally on the shaft 1110, and a guide pulley 1142a is rotatably supported substantially centrally on the shaft 1112. The idle pulley 1140a serves to keep a driven wire 1252a wound around the guide pulley 1142a through a constant angle (about 180° on both sides) at all times. Instead of using the idle pulley 1140a, the driven wire 1252a may be wound one or more turns around the guide pulley 1142a. The idle pulley 1140a and the guide pulley 1142a may have a smooth surface, or may be made of a material having a small coefficient of friction in order to reduce slippage and frictional wear on the driven wire 1252a (see FIG. 11) or the shafts 1110, 1112. The guide pulley 1142a is disposed around the yaw axis Oy of the attitude changing mechanism.

The main shaft 1144 is rotatably supported on the shaft 1112 between the gear body 1126 and the guide pulley 1142a, as well as between the guide pulley 1142a and the gear body 1130. The main shaft 1144 includes a sleeve that projects toward the composite mechanism 1102. The main shaft 1144 has a square hole 1144a defined axially therein. The main shaft 1144 includes two auxiliary plates 1144b disposed on the end thereof in the Z2 direction, for holding both surfaces of the guide pulley 1142a in the Y directions. Each of the auxiliary plates 1144b has holes through which the shaft 1112 extends. The auxiliary plates 1144b are of a chevron shape, which widens progressively in the Z1 direction in order to prevent foreign matter, such as threads or the like, from entering therein.

The composite mechanism 1102 includes an opening/closing mechanism for opening and closing the scissors mechanism 1300, and an attitude changing mechanism for changing the attitude of the scissors mechanism 1300.

The composite mechanism 1102 comprises the gear body 1146, which is rotatably fitted over the circumferential surface of the sleeve of the main shaft 1144, a nut 1148 mounted on a distal end of the main shaft 1144, a transmitting member 1152 having a square cross-sectional shape and an end in the Z2 direction which is inserted in the hole 1144a, a driven pulley (transmitting member) 1156a rotatably supported by a pin 1154 on an end in the Z2 direction of the transmitting member 1152, a driven plate (transmitting member) 1158, and a hollow cylindrical cover (connecting sleeve) 1160.

A thrust bearing 1144c made of resin is disposed on a portion of the main shaft 1144 that abuts against the gear body 1146. Another thrust bearing 1148a made of resin is disposed on a portion of the nut 1148 that abuts against the gear body 1146. The thrust bearings 1144c, 1148a are made of a material having a low coefficient of friction, for reducing wear and torque on the abutting portions and for preventing loads from being directly applied to the face gear 1165. The thrust bearings 1144c, 1148a comprise slide bearings.

The gear body 1146 has a stepped shape comprising a large-diameter portion 1162 that projects in the Z2 direction, a small-diameter portion 1164 that projects in the Z1 direction, and a face gear 1165 disposed on the end of the large-diameter portion 1162 in the Z2 direction. The face gear 1165 is held in mesh with the gears 1134, 1138. The gear body 1146 prevents the nut 1148 from becoming dislodged from the main shaft 1144. The large-diameter portion 1162 has an externally threaded outer circumferential surface.

The driven plate 1158 includes a recess 1166, which is open in the Z2 direction, an engaging cavity 1168 defined in the bottom of the recess 1166, axial ribs 1170 disposed respectively on both surfaces of the driven plate 1158 in the Y directions, and a pair of link holes 1172 defined on both sides of the engaging cavity 1168. The engaging cavity 1168 has a shape that enables engagement with a mushroom-shaped knob 1174 on the distal end of the transmitting member 1152. When the mushroom-shaped knob 1174 engages within the engaging cavity 1168, the driven plate 1158 and the transmitting member 1152 are capable of rotating relatively with respect to each other about the roll axis. The driven plate 1158 has a width substantially equal to the inside diameter of the cover 1160.

The cover 1160 is of a size large enough to cover the composite mechanism 1102 substantially in its entirety, and serves to prevent foreign matter (living tissue, medications, threads, sutures, etc.) from entering into the composite mechanism 1102 and the scissors mechanism 1300.

The cover 1160 has two axial grooves 1175 defined in the inner circumferential surface thereof in diametrically confronting relation to each other. The ribs 1170 of the driven plate 1158 are slidably fitted respectively into the grooves 1175. Further, the cover 1160 includes a pair of bases 1304 disposed on the distal end thereof in mutual confronting relation in the Y directions, and a pair of holes 1307 defined respectively in the bases 1304 near the distal ends thereof. The bases 1304 have respective confronting surfaces, which are flat, for holding the scissors mechanism 1300, a spacer 1340, etc.

The ribs 1170 of the driven plate 1158 are fitted respectively into the grooves 1175 for axially guiding the driven plate 1158. Since the knob 1174 engages within the engaging cavity 1168 of the driven plate 1158, the driven pulley 1156 is axially movable back and forth in the hole 1144a in unison with the driven plate 1158 and the transmitting member 1152, and the driven pulley 1156 can roll about the transmitting member 1152. The cover 1160 is fixed to the large-diameter portion 1162 of the gear body 1146 by threaded engagement, press-fitted engagement, or the like.

The cover 1160 is coupled at a proximal portion thereof to the gear body 1146 (by threaded engagement, press-fitted engagement, welding, or the like). When the gear body 1146 rotates, the cover 1160 and the scissors mechanism 1300 are rotated about the roll axis.

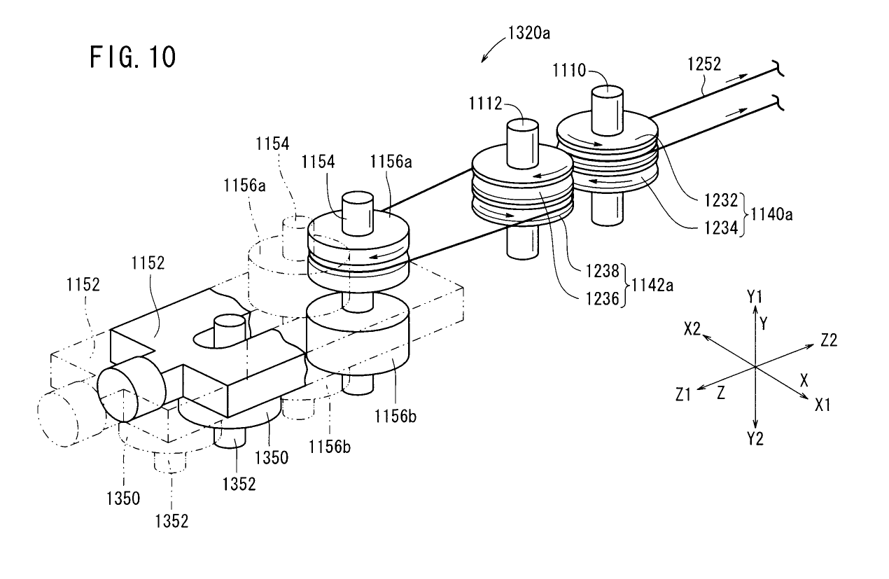

As shown in FIG. 10, the idle pulley 1140a comprises two parallel pulleys, i.e., a first layer idle pulley 1232 and a second layer idle pulley 1234, which are aligned coaxially with each other. Also, the guide pulley 1142a comprises two parallel pulleys, i.e., a first layer guide pulley 1236 and a second layer guide pulley 1238, which are aligned coaxially with each other.

As shown in FIG. 11, the end of the rod 192a in the Z1 direction is connected by a wire engaging member 1250a to both ends of the driven wire 1252a.

The driven wire 1252a comprises a ring-like flexible member having a portion thereof connected to the wire engaging member 1250a. The driven wire 1252a may alternatively comprise a rope, a resin wire, piano wire, a chain, or the like. The term “ring-shaped” should be interpreted in a broad sense. The flexible member is not required to be flexible over its entire length, and at least a portion of the driven wire 1252a, which is trained around each of the pulleys, may consist of a flexible member with a linear portion thereof being connected by a rigid member.

The driven wire 1252a passes from the rod 192a, which serves as a drive member, along the idle pulley 1140a in the X1 direction and proceeds in the X2 direction. The driven wire 1252a then passes along the guide pulley 1142a in the X2 direction and proceeds toward the surface of the driven pulley 1156a in the X2 direction. The driven wire 1252a then is trained one-half turn around the surface of the driven pulley 1156a in the Z1 direction and proceeds toward the surface thereof in the X1 direction, passes along the surface of the guide pulley 1142a in the X1 direction, becomes oriented in the X2 direction, passes along the idle pulley 1140a in the X2 direction, and proceeds toward the wire engaging member 1250a.

The driven wire 1252a thus passes through a circulatory path with starting and ending points thereof at the wire engaging member 1250a. The driven wire 1252a passes along both sides of the idle pulley 1140a, is trained around the driven pulley 1156a, and crosses between the idle pulley 1140a and the guide pulley 1142a, thereby making up a substantially figure-8 configuration. The wire engaging member 1250a and the driven wire 1252a are mechanically connected by the rod 192a to the trigger lever 36.

The idle pulley 1140a, the guide pulley 1142a, and the driven pulley 1156a are of substantially the same diameter, each having as large a diameter as possible given the layout, so that the driven wire 1252a will not be bent excessively. The wire engaging member 1250a is disposed in a position appropriately spaced from the idle pulley 1140a, so that the driven wire 1252a will not be bent excessively. Both ends of the driven wire 1252a form an acute angle at the wire engaging member 1250a. The gap between the idle pulley 1140a and the guide pulley 1142a is small, and for example, is substantially the same as the width of the driven wire 1252a.

The idle pulley 1140a, the guide pulley 1142a, and the driven pulley 1156a may have flanges on upper and lower surfaces thereof, or may have concave side surfaces for preventing the driven wire 1252a from dropping off therefrom.

In the first end effector drive mechanism 1320a, as shown in FIG. 11, the driven wire 1252a, the idle pulley 1140a, the guide pulley 1142a, and the driven pulley 1156a are arranged along a central line from the proximal end toward the distal end. The scissors mechanism 1300 is coupled to the driven pulley 1156a by two links 1220, the driven plate 1158, and the transmitting member 1152, etc.

With the first end effector drive mechanism 1320a, which is constructed in the foregoing manner, when the rod 192a (see FIG. 11) is pulled in the Z2 direction, the first layer idle pulley 1232 and the second layer guide pulley 1238 are rotated counterclockwise as viewed in plan, and the second layer idle pulley 1234 and the first layer guide pulley 1236 are rotated clockwise as viewed in plan. Since the idle pulley 1140a and the guide pulley 1142a each comprises two parallel coaxial pulleys, they are rotatable in opposite directions when the driven wire 1252a held thereagainst is moved, and hence the idle pulley 1140a and the guide pulley 1142a operate smoothly.

As shown in FIGS. 6, 7, 8 and 9, the second end effector drive mechanism 1320b basically is similar to the first effector drive mechanism 1320a (see FIG. 11), except that a return pulley (a cylindrical member, a transmitting member) 1350 is added thereto. The driven pulley 1156a and the driven-pulley 1156b are coaxial with each other.

The main shaft 1144 has a diametrical shaft hole 1354 defined therein with a pin 1352 inserted and fixed in the shaft hole 1354. The shaft hole 1354 extends through the sleeve of the main shaft 1144 and across the hole 1144a.

The transmitting member 1152 has an oblong hole 1356 defined therein, which extends axially and has a width large enough to allow the pin 1352 to be inserted therethrough. The transmitting member 1152 is disposed in a position slightly offset from the axis of the working unit 16 in the Y1 direction, with the knob 1174 on the distal end being disposed on the axis (see FIG. 11). Alternatively, however, the transmitting member 1152 may be centrally positioned.

The pin 1154 extends through the transmitting member 1152 and projects in the Y2 direction, with the driven pulley 1156b being supported on a projecting end. The driven pulley 1156b has a width which is large enough to support two turns of the driven wire 1252b. The hole 1144a has a height large enough to accommodate the driven pulleys 1156a, 1156b and the transmitting member 1152 inserted therein. The driven pulleys 1156a, 1156b are coaxially supported for independent rotation in the hole 1144a by the pin 1154.

Within the hole 1144a, the pin 1352 is inserted through the oblong hole 1356 and the central hole in the return pulley 1350 from the Y1 direction toward the Y2 direction, thus allowing the transmitting member 1152 and the driven pulleys 1156a, 1156b to be moved axially back and forth. The return pulley 1350 is rotatably supported by the pin 1352, is fixed in position, and has a width that is large enough to support two turns of the driven wire 1252b. If the return pulley 1350 is of a two-layer structure, then it can be rotated in opposite directions when the scissors mechanism 1300 is opened and closed, thereby reducing friction between the driven wire 1252b and the pulleys.

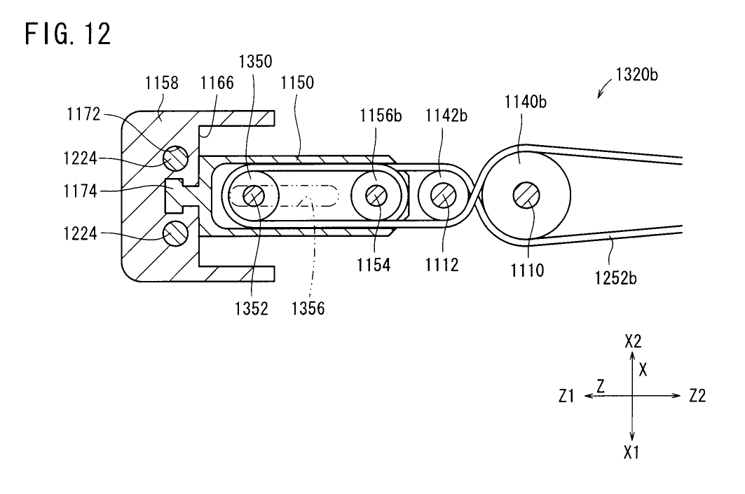

As shown in FIGS. 12, 13 and 14, in the second end effector driving mechanism 1320b, the return pulley 1350 is disposed more closely to the distal end than the driven pulley 1156b, and the driven wire 1252b is trained around the driven pulley 1156b and the return pulley 1350. In other words, the driven wire 1252b passes from the wire engaging member 1250b of the rod 192b, through the side of the idle pulley 1140b that faces in the X1 direction, then proceeds in the X2 direction, passes through the side of the guide pulley 1142b that faces in the X2 direction, and proceeds to the surface of the driven pulley 1156b that faces in the X2 direction. The driven wire 1252b extends in the Z1 direction to the surface of the return pulley 1350 that faces in the X2 direction, is trained one-half turn around the surface of the return pulley 1350 that faces in the X1 direction, and returns in the Z2 direction.

The driven wire 1252b is trained one-half turn around the surface of the driven pulley 1156b that faces in the Z2 direction, passes through a side thereof that faces in the X2 direction, and proceeds again to the return pulley 1350. The driven wire 1252b is trained one-half turn around the surface of the return pulley 1350 that faces in the Z1 direction, and returns in the X2 direction. Thereafter, the driven wire 1252b proceeds from the side of the guide pulley 1142b that faces in the X1 direction to the side of the idle pulley 1140b that faces in the X2 direction, and is connected to the wire engaging member 1250b of the rod 192b. The wire engaging member 1250b and the driven wire 1252b are mechanically connected to the trigger lever 36 by the rod 192b.

FIG. 5 schematically shows the distal-end working unit 12 for facilitating understanding of the structure thereof.

As shown in FIG. 5, when the trigger lever 1032 is fully pulled by the hand, the rod 192a pulls the driven wire 1252a in order to move the transmitting member 1152 in the Z2 direction and close the scissors mechanism 1300. In other words, the scissors mechanism 1300 is closed when the transmitting member, made up of the rod 192a, the driven wire 1252a, the driven pulley 1156a, etc., are pulled.

The scissors mechanism 1300 will be described below.

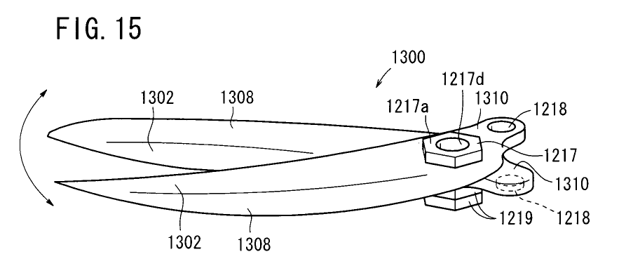

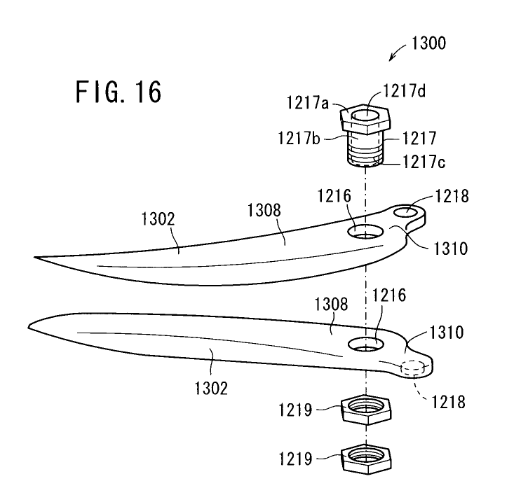

As shown in FIGS. 15 and 16, the scissors mechanism 1300 is in the form of a unit, and is of the double-acting configuration, which comprises a pair of end effector members 1308 with movable cutting blades 1302.

Each of the end effector members 1308 is L-shaped and has a cutting blade 1302 that extends in the Z1 direction, a lever 1310 bent about 35° with respect to the cutting blade 1302, and a shaft hole 1216 defined in an L-shaped bent corner thereof. The end effector member 1308 also has a hole 1218 defined therein near an end thereof. A bolt 1217 is inserted into the shaft hole 1216, whereby the end effector members 1308 are openable and closable about the third rotational axis Og.

The bolt 1217 includes a hexagonal head (head) 1217a, a smooth shaft (shank) 1217b and a threaded portion 1217c that extend from the hexagonal head 1217a, and a central hole (shaft hole) 1217d. The central hole 1217d is defined axially through the bolt 1217. The shaft 1217b has a diameter that enables the shaft 1217b to be fitted in the two shaft holes 1216 with an appropriate tolerance. The shaft 1217b is slightly shorter than the sum of the lengths of the two shaft holes 1216.

The threaded portion 1217c extends through the two shaft holes 1216 and projects toward the other side (in the Y2 direction). Two nuts 1219 are threaded and tightened as double nuts over the projecting end. The scissors mechanism 1300 is assembled by a process (first step) of superposing the end effector members 1308 one on each other, and thereafter inserting the shaft 1217b of the bolt 1217 through the shaft holes 1216 of the end effector members 1308, a process (second step) of threading the nuts 1219 onto the threaded portion 1217c of the tip end of the bolt 1217, and a process (third step) of sandwiching the superposed end effector members 1308 between the hexagonal head 1217a and the nuts 1219 while performing a predetermined sliding adjustment process, and then securing the bolt 1217 and the nuts 1219 to each other in order to bring the cutting blades 1302 into a predetermined sliding state.

The hexagonal head 1217a of the bolt 1217 and the nut 1219 need not necessarily be formed in a hexagonal shape, but may be of a cylindrical shape, a two-faced shape, etc., insofar as they can be tightened to a prescribed torque by a predetermined tool. The double nuts 1219 need not necessarily be tightened on the bolt 1217, but alternatively, a single nut may be tightened on the bolt 1217 and secured by means of a given locking means (e.g., welding, a locking agent, etc.).

The nuts 1219 effectively tighten and secure the superposed end effector members 1308 together with the hexagonal head 1217a strongly and with no gaps therebetween, in order to keep the cutting blades 1302 in a predetermined sliding state. Such a sliding state can be realized by the scissors mechanism 1300 even before it is assembled into the distal-end working unit 12. The scissors mechanism 1300 can thus be assembled highly efficiently by an unskilled worker. The end effector members 1308 can smoothly be turned about the shaft 1217b.

The scissors mechanism 1300 can be assembled at a location with no nearby obstacles. The sliding adjustment process can easily be carried out on the scissors mechanism 1300 while the scissors mechanism 1300 is repeatedly opened and closed, because there are no other interlinking members at this stage. If the scissors mechanism 1300 were placed in the cover 1160 at this stage, then it is easy to understand that it would be difficult to perform the sliding adjustment process, since the space in the cover 1160 is small and other interlinking members, such as the links 1220, the driven plate 1158, etc., are present therein.

The cutting blades 1302 are slightly curved in mutually opposite directions (the Y1 direction and the Y2 direction) when the scissors mechanism 1300 is open. When the scissors mechanism 1300 is closed, therefore, a gap is more effectively prevented from being created between the cutting blades 1302, thus making the scissors mechanism 1300 more effective at cutting an object.

As shown in FIGS. 6 through 8, the scissors mechanism 1300, which is assembled as a unit, is housed in the tubular structure of the cover 1160. The central hole 1217d of the bolt 1217 is disposed coaxially with the two holes 1307, and the pin 1196 is press-fitted and then secured in the holes 1307 and the central hole 1217d. The pin 1196 may be secured at least in the holes 1307 by press-fitting or welding. The end effector members 1308 are angularly movably supported on the pin 1196.

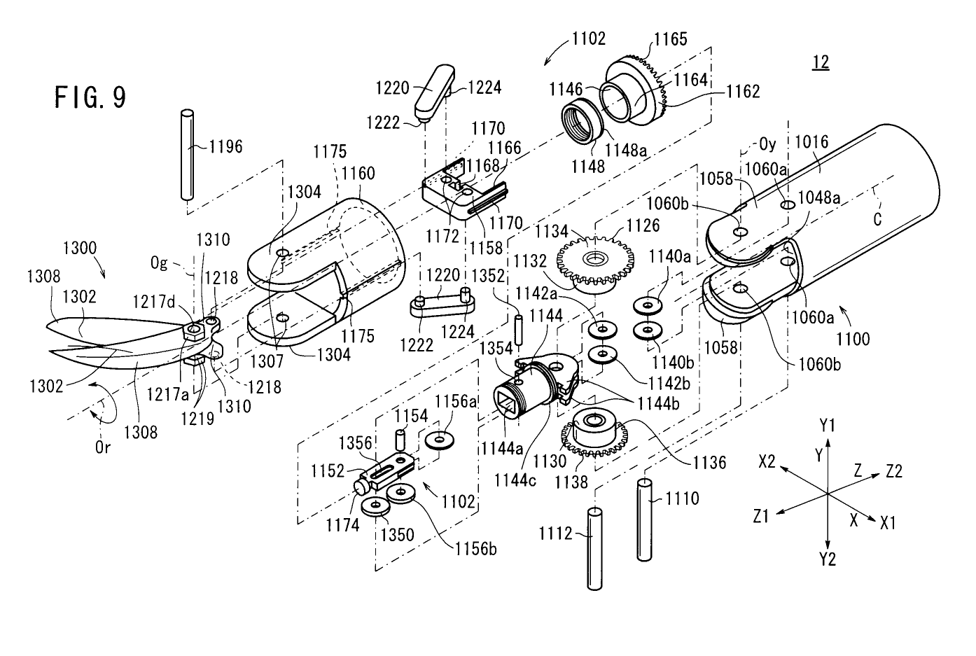

The lever 1310 and the driven plate 1158 are coupled to each other by links 1220 (see FIG. 9). Each of the links 1220 has pins 1222, 1224 near opposite ends thereof, which project in the same direction. The pins 1222, 1224 may be press-fitted into holes defined in the links 1220, so as to project therefrom. The pins 1222 are inserted into the holes 1218, whereas the other pins 1124 are inserted into the link holes 1172 of the driven plate 1158 and are joined thereto.

A spacer 1340 through which the pin 1196 extends is disposed in the cover 1160 and placed in a gap between the scissors mechanism 1300 and an inner surface of the cover 1160 that faces in the Y2 direction. The spacer 1340 has a hole 1340a defined therein, which is open in the Z2 direction, while keeping clear of the pin 1196.

The spacer 1340, which is sandwiched between the scissors mechanism 1300 and the inner surface of the cover 1160, effectively prevents the scissors mechanism 1300 from being positionally displaced in the cover 1160.

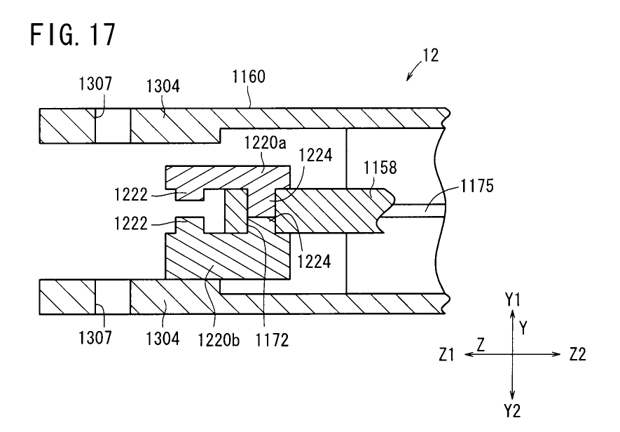

A process for assembling and manufacturing the distal-end working unit 12 will be described below. When the distal-end working unit 12 is assembled, it is assumed that the scissors mechanism 1300 has already been assembled together as a unit (see FIG. 15). It also is assumed that, except for the cover 1160, the driven plate 1158 and other parts which are closer to the distal end than the driven plate 1158 have been assembled, and that the pins 1224 of the links 1220 have been inserted into the two link holes 1172 of the driven plate 1158 from opposite directions. In FIGS. 17 through 25, among the two links 1220, the link positioned in the Y1 direction is referred to as a link 1220a, whereas the link positioned in the Y2 direction is referred to as a link 1220b. Among the end effector members 1308, the end effector member positioned in the Y1 direction is referred to as an end effector member 1308a, whereas the end effector member positioned in the Y2 direction is referred to as an end effector member 1308b. The distal-end working unit 12 is illustrated schematically in FIGS. 17 through 25 for facilitating understanding. Although the two link holes 1172 actually are displaced from each other in the X directions, they are illustrated as lying in one sectional plane.

As shown in FIG. 17, the driven plate 1158 and the links 1220 are covered by the cover 1160. At this time, the ribs 1170 are fitted into the grooves 1175 of the cover 1160 to guide the cover 1160 in a suitable direction. As described above, the cover 1160 is fixed to the large-diameter portion 1162 of the gear body 1146.

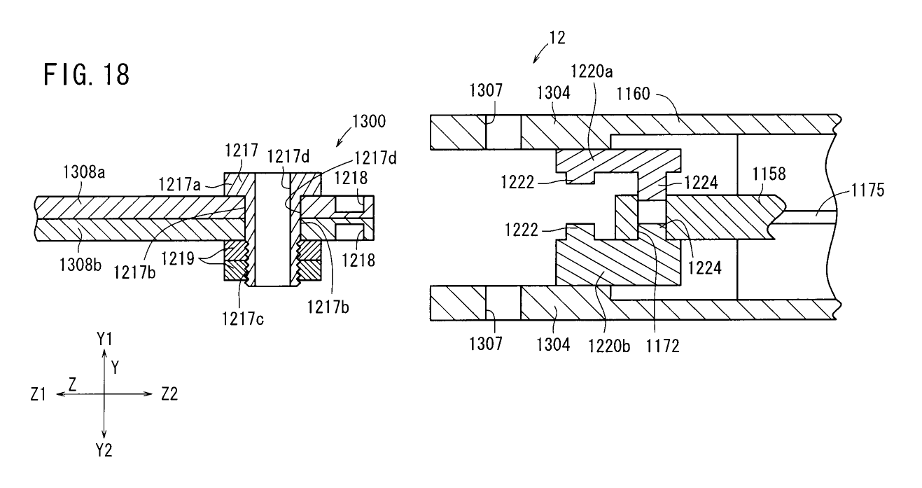

Then, as shown in FIG. 18, the scissors mechanism 1300 is held in a suitable direction near the cover 1160, while the link 1220a is lifted in the Y1 direction. Since the pin 1224 of the link 1220a is inserted somewhat deeply into the link hole 1172, and since the space in the cover 1160 is small, the pin 1224 is prevented from becoming dislodged from the link hole 1172.

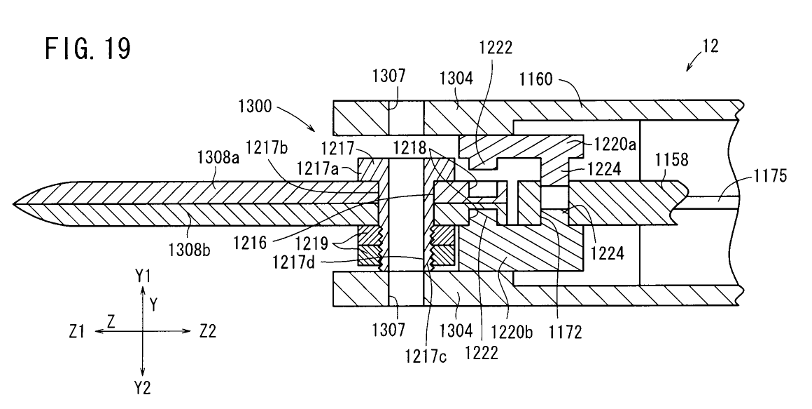

Then, as shown in FIG. 19, the proximal end portion of the scissors mechanism 1300 is inserted into the cover 1160 in the Z2 direction. The pin 1222 of the link 1220b is inserted into the hole 1218 of the end effector member 1308b, and the central hole 1217d of the bolt 1217 is positioned coaxially with the holes 1307 of the cover 1160 (fourth step).

At this time, the scissors mechanism 1300 may be inserted along a side surface of the cover 1160, which faces away from the Y1 direction, and then may be lowered in the Y2 direction when the scissors mechanism 1300 reaches a suitable inserted position. The link 1220a may be angularly moved in a suitable direction about the pin 1224, so as to keep the link 1220a out of engagement with the scissors mechanism 1300. The above series of operations may be carried out by a finger, or by a tool that is inserted between the bases 1304 from the space defined therebetween, which is exposed in the X directions. The tool may simply be a general tool as well, such as a pair of tweezers.

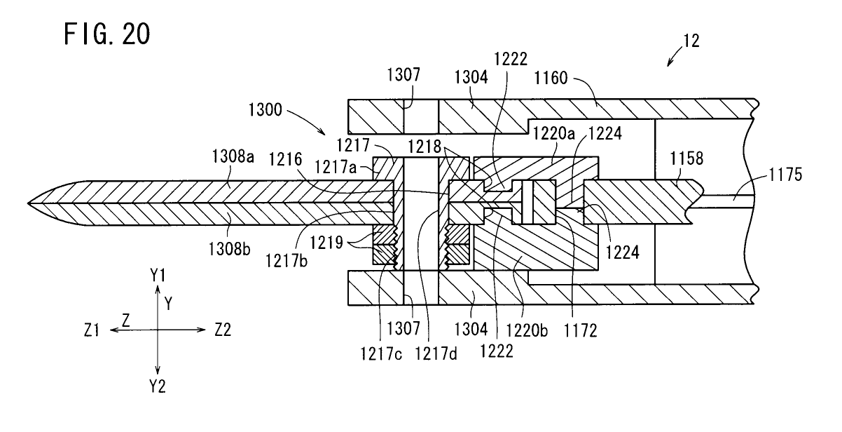

Then, as shown in FIG. 20, after the link 1220a has been directed in a suitable orientation, the link 1220a is lowered in the Y2 direction, and the pin 1222 thereof is inserted into the hole 1218 of the end effector member 1308a (fourth step).

The process (fourth step) of inserting the scissors mechanism 1300 into the tubular structure of the cover 1160, and connecting the end effector members 1308 to the driven plate 1158 that serves as the transmitting member, need not strictly be carried out in the above sequence, insofar as the scissors mechanism 1300 can be assembled from the state shown in FIG. 18 to the state shown in FIG. 19.

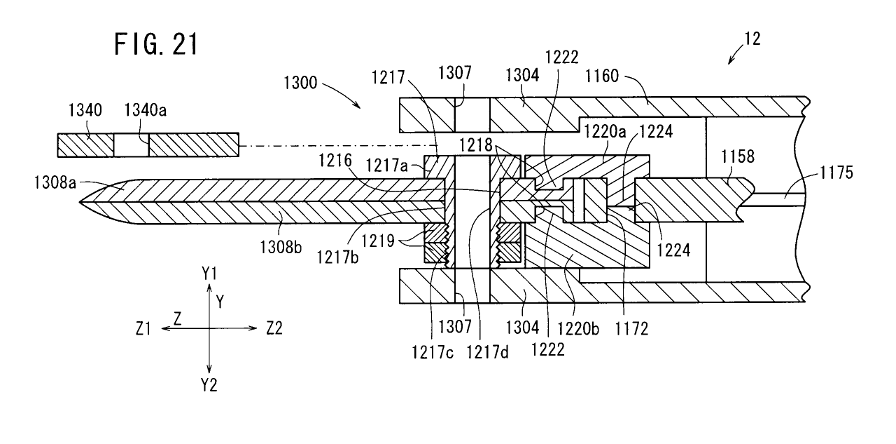

Then, as shown in FIG. 21, the spacer 1340 is inserted in the Z2 direction into the space that has been created between the cover 1160 on the side of the scissors mechanism 1300 and the link 1220a which faces in the Y1 direction (spacer insertion step). With the spacer 1340 thus inserted, the scissors mechanism 1300 is prevented from wobbling, and the pins 1222, 1224 of the link 1220a are prevented from becoming dislodged from the hole 1218 and the link hole 1172. The spacer 1340 is disposed such that the hole 1340a thereof is positioned directly below the hole 1307 of the cover 1160.

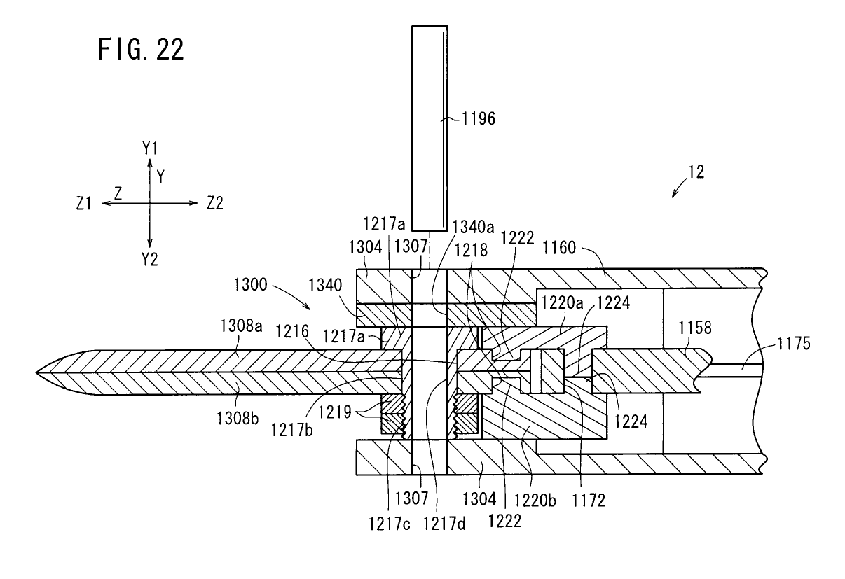

Finally, as shown in FIG. 22, the pin 1196 is press-fitted and then secured in the holes 1340a, 1307 and the central hole 1217d, thereby supporting the end effector members 1308 for angular movement (fifth step).

With the distal-end working unit 12 thus assembled and manufactured, since the scissors mechanism 1300 has already been assembled as a unit, the cutting blades 1302 of the scissors mechanism 1300 is maintained in a state of sliding adjustment, thereby enabling the scissors mechanism 1300 to cut an object effectively. Insofar as dynamic sliding adjustment does not need to be carried out within a small space inside the cover 1160, the scissors mechanism 1300 can be assembled in the cover 1160 easily even by an unskilled worker.

A process for assembling and manufacturing a distal-end working unit 12a, which forms a first modification of the distal-end working unit 12, will be described below with reference to FIGS. 23 through 25.

The distal-end working unit 12a includes a hexagonal head 1217a and links 1220a, 1220b, which are relatively thick in the Y directions. Therefore, the scissors mechanism 1300 is stably held in the cover 1160 in the absence of the spacer 1340, and the pins 1222, 1224 of the links 1220a, 1220b are prevented from becoming dislodged from the holes 1218 and the link holes 1172. The height of the bolt 1217 may be set depending on the distance between the bases 1304, or conversely, the distance between the bases 1304 may be reduced. For the sake of brevity, components of the distal-end working unit 12a, which are identical to those of the distal-end working unit 12, are denoted by identical reference characters, and such features will not be described in detail below.

As shown in FIG. 23, the scissors mechanism 1300 is held in a suitable direction near the driven plate 1158. The links 1220a, 1220b are mounted on the scissors mechanism 1300.

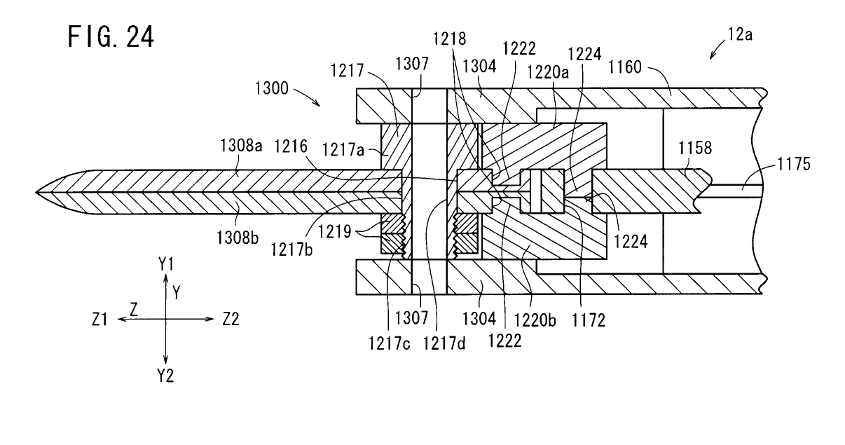

Then, as shown in FIG. 24, the proximal end of the scissors mechanism 1300, the driven plate 1158, and the links 1220 are covered by the cover 1160. At this time, the ribs 1170 are fitted into the grooves 1175 of the cover 1160 to guide the cover 1160 in a suitable direction. The cover 1160 is disposed such that the central hole 1217d of the bolt 1217 is coaxial with the hole 1307 of the cover 1160.

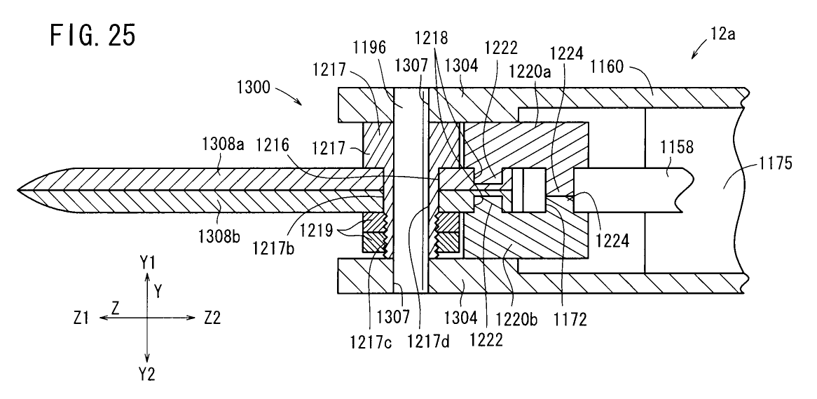

Finally, as shown in FIG. 25, the pin 1196 is press-fitted and then secured in the hole 1307 and the central hole 1217d, thereby supporting the end effector members 1308 for angular movement.

With the distal-end working unit 12a and the process of assembling and manufacturing the same, in contrast to the distal-end working unit 12, the spacer 1340 is omitted. Therefore, the distal-end working unit 12a is simpler in structure and can be assembled more easily than the distal-end working unit 12.

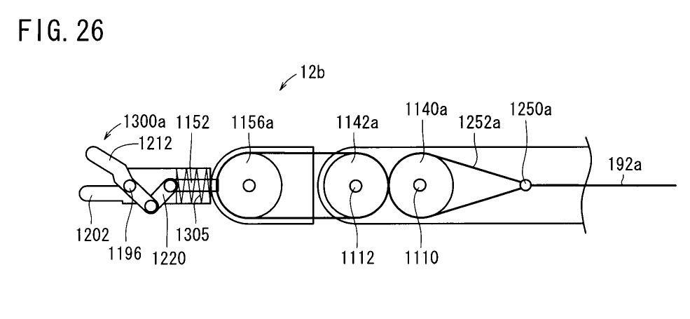

FIG. 26 shows a distal-end working unit 12b, which constitutes a second modification of the distal-end working unit 12.

As shown in FIG. 26, the distal-end working unit 12b is similar to the distal-end working unit 12, in that it includes the first end effector drive mechanism 1320a. However, the distal-end working unit 12b differs from the distal-end working unit 12 in that it lacks the second end effector drive mechanism 1320b.

The distal-end working unit 12b comprises a single-acting type scissors mechanism 1300a, instead of the double-acting type scissors mechanism 1300. The scissors mechanism 1300a comprises a fixed cutting blade 1202, a movable cutting blade 1212 closable toward and openable away from the fixed cutting blade 1202 about the pin 1196, and a spring 1305, which normally urges the transmitting member 1152 to move in the Z1 direction. The movable cutting blade 1212 can be closed toward or opened away from the fixed cutting blade 1202 by the link 1220, which is actuated when the transmitting member 1152 is displaced. More specifically, when the trigger lever 36 is pulled in the Z2 direction, the transmitting member 1152 is displaced in the Z2 direction by the first end effector drive mechanism 1320a, thereby turning the movable cutting blade 1212 counterclockwise in FIG. 26 to close the scissors mechanism 1300a. When the trigger lever 36 is opened, the transmitting member 1152 is displaced in the Z1 direction under the resiliency of the spring 1305 in order to return the scissors mechanism 1300a to an open state. The trigger lever 36 is also returned in the Z1 direction.

The scissors mechanism 1300a may be preassembled as a unit, in the same manner as the scissors mechanism 1300. Therefore, the scissors mechanism 1300a has the cutting blades 1202, 1212 thereof held in a state of an appropriate sliding adjustment, and can be assembled with ease.

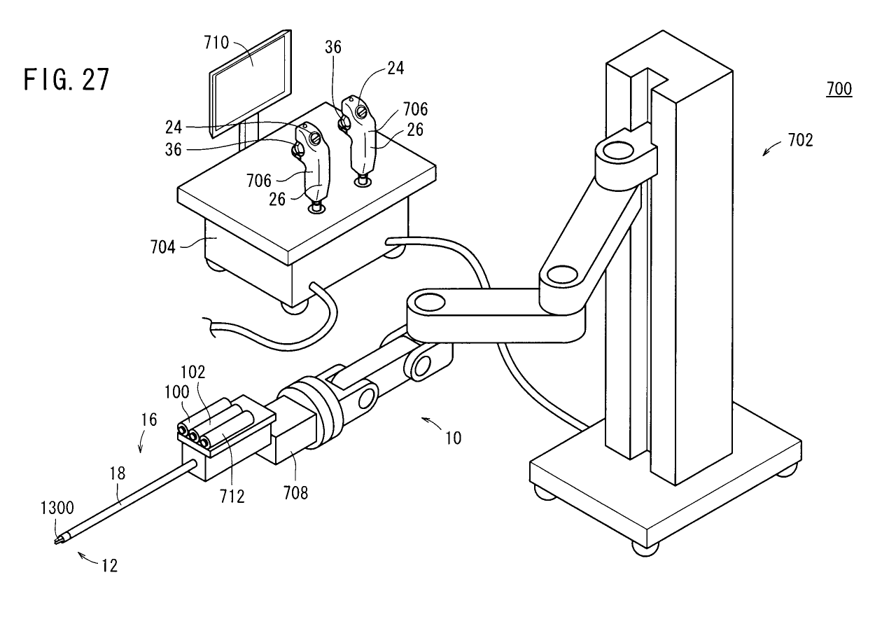

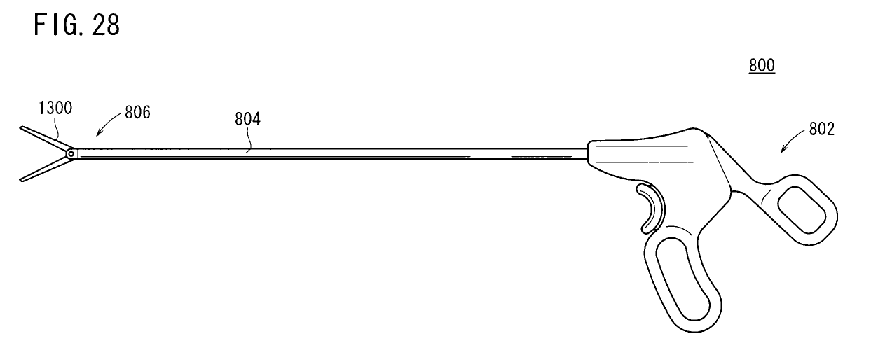

The distal-end working units 12, 12a, 12b, which incorporate the scissors mechanisms 1300, 1300a therein, may be applied to a surgical robot system 700 as shown in FIG. 27, or to a pair of forceps 800 as shown in FIG. 28, for example.

As shown in FIG. 27, the surgical robot system 700 has an articulated robot arm 702 and a console 704, with the working unit 16 connected to the distal end of the robot arm 702. The distal end of the robot arm 702 incorporates therein a mechanism which functions the same as the manipulator 10. The robot arm 702 may constitute a means for moving the working unit 16, and is not limited to an installed type, but may be an autonomous movable type. The console 704 may be a table type, a control panel type, or the like.

The robot arm 702 should preferably have six or more independent joints (rotary shafts, slide shafts, etc.) for setting the position and orientation of the working unit 16 as desired. The manipulator 10 on the distal end of the robot arm 702 is integrally combined with the distal end 708 of the robot arm 702. The manipulator 10 includes a motor 712 instead of the trigger lever 36 (see FIG. 1). The motor 712 actuates the two rods 192a, 192b.

The robot arm 702 operates under the control of the console 704, and may be actuated automatically according to a program, or by joysticks 706 mounted on the console 704, or by a combination of a program and the joysticks 706. The console 704 includes the function of the controller 11. The working unit 16 includes the distal-end working unit 12, including the scissors mechanism 1300.

The console 704 includes the two joysticks 706 that serve as an operation commander, and a monitor 710. Although not shown, the two joysticks 706 are capable of individually operating two robot arms 702. The two joysticks 706 are disposed in respective positions where they can easily be operated by both hands of the operator. The monitor 710 displays information such as an image produced by a flexible scope.

The joysticks 706 can be moved vertically and horizontally, twisted, and tilted, whereby the robot arm 702 can be moved depending on movements of the joysticks 706. The robot arm 702 and the console 704 may be connected to each other by a communicating means, such as a wired link, a wireless link, a network, or a combination thereof.

The joysticks 706 have respective trigger levers 36, which can be operated in order to energize the motor 712.

As shown in FIG. 28, the forceps 800 are basically of a conventional structure, which is free of an electric actuator, and incorporate the scissors mechanism 1300 therein. The forceps 800 include a hand operating unit 802, a shaft 804 having a small diameter extending from the hand operating unit 802, and a distal-end working unit 806. The scissors mechanism 1300 is incorporated in the distal-end working unit 806. The hand operating unit 802 comprises a pair of handles, which can be opened and closed by fingers inserted therein. When the handles are opened and closed, the scissors mechanism 1300 is opened and closed accordingly.

The scissors mechanisms 1300, 1300a can also be applied to the distal end portion (connecting tube) of an endoscope (medical instrument), for example.

Although certain preferred embodiments of the present invention have been shown and described in detail, it should be understood that various changes and modifications may be made to the embodiments without departing from the scope of the invention as set forth in the appended claims.

What is claimed is:

1. A method of manufacturing a medical instrument, comprising:
a first step of superposing a pair of openable and closable members one on each other, and inserting a shank of a bolt through holes defined in proximal ends of the openable and closable members, the bolt having a shaft hole defined axially through the bolt;
a second step of threading a nut on the shank of the bolt;
a third step of sandwiching the superposed openable and closable members between a head of the bolt and the nut, and while performing a predetermined sliding adjustment process on the superposed openable and closable members, securing the bolt and the nut to each other to produce a structural body in which the openable and closable members are angularly movably supported in a predetermined sliding state;
a fourth step of inserting the structural body into a connecting tube coupled to a distal end of a shaft extending from an operating unit, and connecting the openable and closable members to a transmitting member for transmitting an input action from the operating unit to the openable and closable members; and
a fifth step of fitting and securing a pin, from an outer surface of the connecting tube, in a hole defined diametrically through the connecting tube and the shaft hole of the bolt, whereby the openable and closable members are angularly movably supported on the shank of the bolt for opening and closing movement about the shank.

2. A method according to claim 1, further comprising:
after the fourth step and before the fifth step, a spacer insertion step of placing a spacer in a gap between the structural body and an inner surface of the connecting tube.

3. A medical instrument comprising:
a structural body including a pair of superposed openable and closable members fastened to each other in a predetermined sliding state by a bolt and a nut, the superposed openable and closable members being openable and closable about a shank of the bolt;
a connecting tube housing the structural body therein and coupled to a distal end of a shaft extending from an operating unit;
a pin fitted into a shaft hole defined axially through the bolt in the structural body which is housed in the connecting tube, the openable and closable members being angularly movably supported by the pin; and
a transmitting member for transmitting an input action from the operating unit to the openable and closable members.

4. A medical instrument according to claim 3, further comprising:
a spacer disposed in the connecting tube and placed in a gap between the structural body and an inner surface of the connecting tube, the pin being inserted in the spacer.

5. A medical instrument according to claim 3, wherein the openable and closable members comprise a pair of scissors held in sliding contact with each other for cutting an object.
